(19) United States
(12) Patent Application Publication
(10) Pub. No.: US 2012/0266876 A1
(43) Pub. Date: Oct. 25, 2012

(54) Medicament Container

(75) Inventors: Thomas Nagel, Tharandt (DE); Rene Richter, Tharandt (DE); Robert Witt, Dresden (DE)

(73) Assignee: SANOFI-AVENTIS DEUTSCHLAND GMBH, Frankfurt am Main (DE)

(21) Appl. No.: 13/392,736

(22) PCT Filed: Aug. 20, 2010
(86) PCT No.: PCT/EP2010/062155

§ 371 Date: Jul. 10, 2012

(30) Foreign Application Priority Data
Sep. 2, 2009 (EP) 09011255.8

(51) Int. Cl. A61M 5/31; A61M 5/44; A61M 15/00; A61M 5/00

(52) U.S. Cl. 128/203.12; 604/257; 604/113; 604/213

(57) ABSTRACT

The invention relates to a medicament container (2), comprising a bag (3) with an outlet (4), the bag (3) compressible by a compression means (6), the bag (3) having an inner surface arranged to stick when subjected to the compression means (6). The invention also refers to an arrangement (1) for administering a liquid medicament (M) comprising the medicament container (2) and a compression means (6) for locally compressing the bag (3).

### TECHNICAL FIELD

The invention relates to a medicament container, comprising a bag with an outlet, the bag compressible by a compression means.

### BACKGROUND OF THE INVENTION

Many medicaments have to be injected into the body. This applies in particular to medicaments, which are deactivated or have their efficiency remarkably decreased by oral administration, e.g. proteines (such as insulin, growth hormones, interferons), carbohydrates (e.g. heparin), antibodies and the majority of vaccines. Such medicaments are predominantly injected by means of syringes, medicament pens or medicament pumps.

Some medicaments have to be administered by inhaling them from so called inhalers.

WO 2009/069518 A1 discloses an inhaler, wherein the medicament to be inhaled is stored in a bag shaped medicament container.

### SUMMARY OF THE INVENTION

It is an object of the present invention to provide an arrangement for administering a liquid medicament comprising a medicament container with a bag, further comprising a compression means for locally compressing the bag, wherein the bag has an outlet and an inner surface, wherein the arrangement avoids intake of air or reflow of remaining medicament into an emptied area of the bag when the compression means is pulled back from the bag after delivering a dose of medicament.

The object is achieved by an arrangement according to claim 1.

Preferred embodiments of the invention are given in the dependent claims.

According to the invention an arrangement for administering the liquid medicament comprises a medicament container with a bag. Furthermore the arrangement comprises a compression means for locally compressing the bag. The medicament container according to the invention comprises a flexible bag with an outlet. The bag, preferably having a relatively thin skin, is compressible by a the compression means so as to gradually reducing a volume of the bag and consequently squeezing the medicament stored in the bag out of the outlet similar to a tube of tooth paste. The bag is provided with an inner surface arranged to stick when subjected to the compression means. Thus the bag is kept from returning to its initial shape after having been subjected to the compression means so intake of air or reflow of the remaining medicament into the emptied area is avoided when the compression means is pulled back from the bag after delivering a dose of medicament. It is therefore not required to maintain the pressure exerted by the compression means after delivering a dose as opposed to conventional systems with rollers for compressing the bag and performing an advancing motion thereby sealing the portion of the bag already compressed against the portion not yet compressed.

In one embodiment of the invention the inner surface of the bag is self-adhesive so opposing surface parts stick together when brought in contact by the compression means.

In another embodiment of the invention the inner surface is arranged to be welded under pressure which may be exerted by the compression means.

In yet another embodiment of the invention the inner surface is arranged to be welded by heat. The heat may be introduced by a heated compression means.

The medicament container may be part of an injection arrangement or an inhaler arrangement for delivering a liquid medicament to a human or an animal, wherein the arrangement comprises the compression means for locally compressing the bag. The injection arrangement may comprise a valve and a hollow needle for piercing a patient's skin, the valve and needle being arranged at the outlet of the medicament container. In case of a jet injector, instead of the needle, a jet nozzle may be arranged.

In a preferred embodiment of the invention the bag is arranged on an essentially planar, rigid support and a roller is arranged for locally pressing the bag against the support and advancing in a longitudinal direction of the bag. The roller may alternatively be replaced by a shoe or wiper or ram pressed against the bag.

The ram may be used to consecutively compressing, emptying and sealing a portion of the bag. The ram may be operated overlapping an edge of the bag in order to achieve delivery of a dose smaller than corresponding to an outline of the ram when placed in the middle of the bag.

The compression means may be arranged to introduce heat into the bag in order to achieve a heat-induced welding of the inner surface.

The injection arrangement may comprise a valve and a hollow needle for piercing a patient's skin, the valve and needle being arranged at the outlet of the medicament container. In case of a jet injector, instead of the needle, a jet nozzle may be arranged.

The medicament container may preferably be used for delivering one of an analgetic, an anticoagulant, insulin, an insulin derivate, heparin, Lovenox, a vaccine, a growth hormone, a peptide hormone, a proteine, antibodies and complex carbohydrates.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is a schematic view of an arrangement for administering a liquid medicament comprising a medicament container with a bag compressible by a compression means in the shape of a roller, and

FIG. 2 is a schematic view of another embodiment of the arrangement with the compression means in the shape of a ram.

Corresponding parts are marked with the same reference symbols in all figures.

### DETAILED DESCRIPTION OF PREFERRED EMBODIMENTS

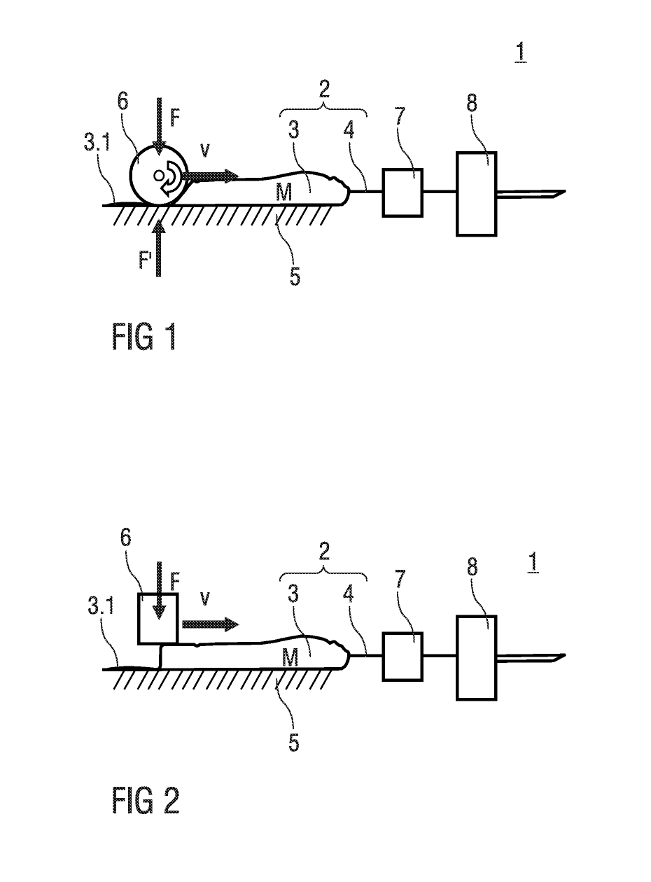

FIG. 1 shows a schematic view of an arrangement 1 for administering a liquid medicament M comprising a medicament container 2. The medicament container 2 comprises a flexible bag 3 with an outlet 4. The bag 3 is arranged on an essentially planar, rigid support 5. The bag 3, preferably having a relatively thin skin, is compressible by a compression means 6 so as to gradually reducing a volume of the bag 3 and consequently squeezing the medicament M stored in the bag 3 out of the outlet 4. The bag 3 is provided with an inner surface arranged to stick when subjected to the compression means 6. When being compressed by the compression means 6, opposing parts of the inner surface are pressed together and consequently stick. Hence, after having been compressed by the compression means 6 the bag 3 is kept from returning to its initial shape in an already emptied portion 3.1.

The compression means 6 shown in FIG. 1 has the shape of a roller. The roller 6 is arranged for locally pressing the bag 3 against the support 5 with a force F while the support 5 exerts a counteracting force F′. In order to squeeze the medicament M out of the bag 3 the roller 6 is advanced in a longitudinal direction of the bag 3 with a speed v.

The inner surface of the bag 3 may be self-adhesive or arranged to be welded under pressure which may be exerted by the compression means 6.

The inner surface may also be arranged to be welded by heat. The heat may be introduced by a heated compression means 6.

The arrangement 1 shown in FIG. 1 is an injection arrangement comprising a valve 7 and a hollow needle 8 for piercing a patient's skin, the valve 7 and needle 8 being arranged at the outlet 4 of the medicament container 2. The arrangement 1 may alternatively be a jet injector with a jet nozzle instead of the needle 8.

The medicament container 2 may be part of another arrangement 1 for administering the liquid medicament M, e.g. an inhaler arrangement for delivering a liquid medicament M to a human or an animal.

FIG. 2 is a schematic view of another embodiment of the arrangement 1 which differs from the embodiment shown in FIG. 1 by the compression means 6 having the shape of a ram. The ram 6 may be used to consecutively compressing, emptying and sealing a portion of the bag 3. FIG. 2 shows the ram 6 being operated overlapping an edge of the bag 3 in order to achieve delivery of a dose smaller than corresponding to an outline of the ram when placed in the middle of the bag 3 or in another non-overlapping manner. The edge may be a lateral edge or an edge of the bag 3 towards the already emptied portion 3.1 as shown in FIG. 2.

The compression means 6 may also be a shoe or wiper pressed against the bag 3.

The medicament container 2 or the arrangement 1 may preferably be used for delivering one of an analgetic, an anticoagulant, insulin, an insulin derivate, heparin, Lovenox, a vaccine, a growth hormone, a peptide hormone, a proteine, antibodies and complex carbohydrates.

The term “medicament”, as used herein, means a pharmaceutical formulation containing at least one pharmaceutically active compound,
wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(w-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group-Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### LIST OF REFERENCES

1 arrangement for administering a liquid medicament

2 medicament container

3 flexible bag

3.1 already emptied portion

4 outlet

5 rigid support

6 compression means

7 valve

8 hollow needle

F force

F′ counteracting force

M medicament

v speed

1. Arrangement for administering a liquid medicament comprising a medicament container with a bag, further comprising a compression means for locally compressing the bag, wherein the bag has an outlet and an inner surface, characterized in that the inner surface is arranged to stick when subjected to the compression means and thus kept from returning to its initial shape after having been subjected to the compression means.

2. Arrangement according to claim 1, characterized in that the inner surface is self-adhesive.

3. Arrangement according to claim 1, characterized in that the inner surface is arranged to be welded under pressure.

4. Arrangement according to claim 1, characterized in that the inner surface is arranged to be welded by heat or by subjection to ultrasonic energy.

5. Arrangement according to claim 1, characterized in that the bag is arranged on an essentially planar, rigid support.

6. Arrangement according to claim 1, characterized in that the bag is arranged between two opposing parts of the compression means.

7. Arrangement according to claim 5, characterized in that the compression means is arranged for locally pressing the bag against the support and advancing at least in a longitudinal direction of the bag.

8. Arrangement according to claim 1, characterized in that the compression means comprises at least one of a roller, a wiper, a shoe and a ram.

9. Arrangement according to claim 6, characterized in that the compression means comprises two rollers pressing against each other.

10. Arrangement according to claim 4, characterized in that the compression means is arranged to introduce heat into the bag.

11. Arrangement according to claim 4, characterized in that the compression means is arranged to introduce ultrasonic energy into the bag.

12. Arrangement according to claim 1, arranged to be an injection arrangement or an inhaler arrangement.

13. Arrangement according to claim 12, characterized in that a valve and a hollow needle for piercing a patient's skin are arranged at the outlet.
